(19) United States
(12) Patent Application Publication
(10) Pub. No.: US 2026/0326033 A1
(43) Pub. Date: Oct. 22, 2026

(54) Air Purifier & Photocatalytic Filter Cartridge

(71) Applicant: EcoFresh Innovations LLC, Portland, OR (US)

(72) Inventors: William James Kelly, Portland, OR (US); David Demots, Portland, OR (US); Ian Coats MacColl, Mill Valley, CA (US); Jeff Waldo Sand, San Francisco, CA (US); Nicholas Upton Day, Portland, OR (US); Joshua Dylan Olsen, Portland, OR (US); Dillon James Kelly, Nanning City (CN)

(73) Assignee: EcoFresh Innovations LLC, Portland, OR (US)

(21) Appl. No.: 19/643,900

(22) Filed: Apr. 10, 2026

Related U.S. Application Data
(60) Provisional application No. 63/790,063, filed on Apr. 17, 2025.

(51) Int. Cl. B01D 53/88; B01D 46/00; B01D 46/62; B01D 53/00

(52) U.S. Cl. CPC B01D 53/885; B01D 46/0047; B01D 46/009; B01D 46/0091; B01D 46/62; B01D 53/007; B01D 2255/802; B01D 2257/708; B01D 2257/90; B01D 2257/91; B01D 2258/06; B01D 2259/802; B01D 2259/804; B01D 2273/30

(57) ABSTRACT

An air purifier has a photocatalytic filter cartridge that filters and purifies air. The photocatalytic filter cartridge has a middle photocatalytic filter bin, a cover that snaps onto the middle photocatalytic filter bin, and a bottom tray that snaps onto the middle photocatalytic filter bin. Inside the middle photocatalytic filter bin is a pre-filter, a HEPA secondary filter, and photocatalytic material. The air purifier has a light source that shines light (e.g., UV or visible) into the photocatalytic filter cartridge to illuminate the photocatalytic material. The air purifier also has a fan assembly that draws air into and through the photocatalytic filter cartridge. As the air flows through the photocatalytic filter cartridge, the photocatalytic filter cartridge filters and chemically purifies the air.

### CROSS-REFERENCE TO RELATED APPLICATION

This patent application claims the benefit of U.S. Provisional Application 63/790,063 filed Apr. 17, 2025 and incorporated herein by reference in its entirety.

### BACKGROUND

The subject matter described herein generally relates to air purification and, more particularly, the subject matter relates to disinfection, sterilization, or deodorization of air, to air separation and filtering, and to the use of air currents for screening.

Conventional air purifiers only marginally improve indoor air quality. Conventional air purifiers move air through a filter to capture some airborne particles, such as dust, pollen, pet dander, mold, and bacteria. While conventional air filtration schemes can remove some airborne particles, much improvement is needed.

### SUMMARY

An air purifier has a replaceable, serviceable photocatalytic filter cartridge that filters and purifies air. The photocatalytic filter cartridge has a middle photocatalytic filter bin, a cover that snaps onto the middle photocatalytic filter bin, and a bottom tray that snaps onto the middle photocatalytic filter bin. Inside the middle photocatalytic filter bin is a pre-filter, a HEPA secondary filter, and photocatalytic material. The air purifier has a light source that shines light (e.g., UV or visible) into the photocatalytic filter cartridge to illuminate the photocatalytic material. The air purifier also has a fan assembly that draws air into and through the photocatalytic filter cartridge. As the air flows through the photocatalytic filter cartridge, the photocatalytic filter cartridge filters and chemically purifies the air. Should the photocatalytic filter cartridge degrade, the photocatalytic filter cartridge may be returned to a service center for refurbishment and replacement.

### BRIEF DESCRIPTION OF THE SEVERAL VIEWS OF THE DRAWINGS

The features, aspects, and advantages of an air purifier and a photocatalytic filter cartridge are understood when the following Detailed Description is read with reference to the accompanying drawings, wherein:

FIGS. 1-3 illustrate some exterior examples of an air purifier;

FIGS. 4-5 illustrate some interior, sectional examples of the air purifier;

FIG. 6 illustrates sectional examples of tan upper photocatalytic purification assembly;

FIGS. 7-10 illustrate exploded assembly examples of the air purifier;

FIGS. 11-13 illustrate examples of electromechanical safety features;

FIGS. 14-22 illustrate examples of servicing the photocatalytic filter cartridge;

FIGS. 22-23 illustrate more examples of more photocatalytic features;

FIG. 24 illustrates examples of a QR-based lifecycle tracking system;

FIG. 25 illustrates examples of a fused filter component; and

FIGS. 26-27 illustrate examples of methods and operations for centralized factory refurbishment of the air purifier and/or the photocatalytic filter cartridge.

### DETAILED DESCRIPTION

An air purifier and a photocatalytic filter cartridge will now be described more fully hereinafter with reference to the accompanying drawings. The air purifier and the photocatalytic filter cartridge, however, may be embodied in many different forms and should not be construed as limited to the examples set forth herein. These examples are provided so that this disclosure will be thorough and complete and fully convey air purifier and the photocatalytic filter cartridge to those of ordinary skill in the art. Moreover, all the examples of air purifier and the photocatalytic filter cartridge are intended to encompass both structural and functional equivalents thereof. Additionally, it is intended that such equivalents include both currently known equivalents as well as equivalents developed in the future (i.e., any elements developed that perform the same function, regardless of structure).

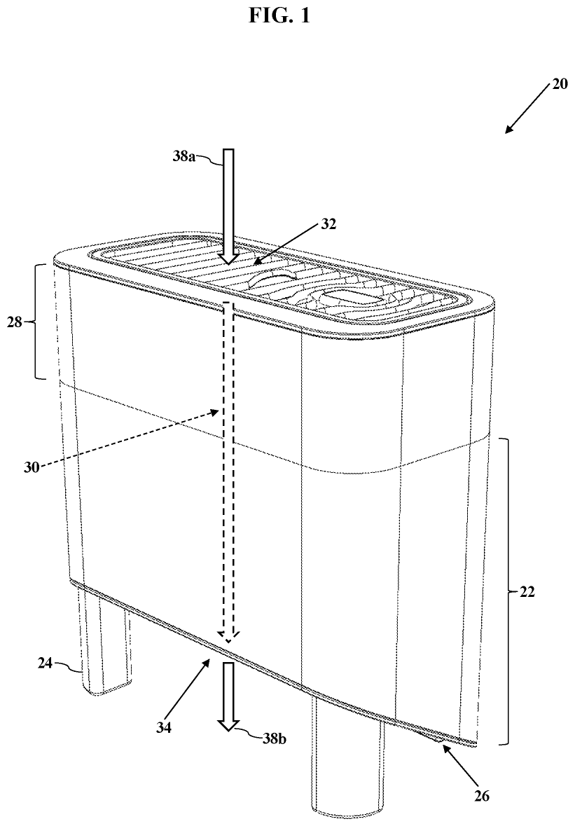

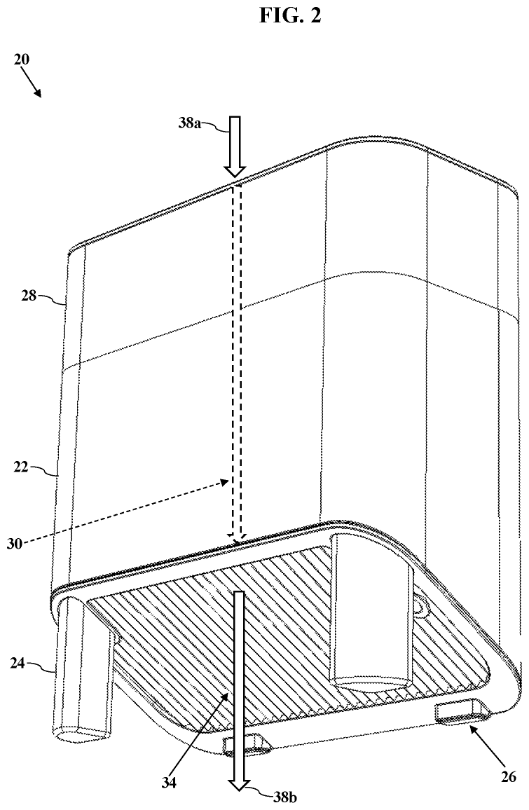

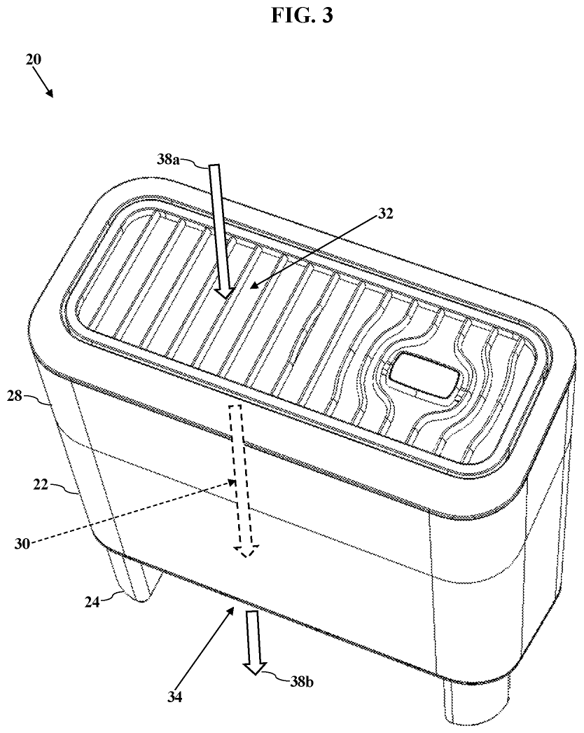

FIGS. 1-3 illustrate some exterior examples of an air purifier 20. The air purifier 20 has a lower, main housing unit 22 with stability legs 24 and feet 26. The air purifier 20 has an upper photocatalytic purification assembly 28 that removably mates with the lower, main housing unit 22. The air purifier 20 has an internal airflow path 30 from an intake or inlet 32 into and through the photocatalytic purification assembly 28 to an outlet 34 in the lower housing unit 22. The lower housing unit 22 has an internal fan assembly 36 (not shown for simplicity) that draws ambient air 38a through the inlet 32 and into the photocatalytic purification assembly 28. As the ambient air 38a enters and flows through the photocatalytic purification assembly 28, the photocatalytic purification assembly 28 produces filtered and purified air 38b (as later paragraphs will explain). The internal fan assembly 36 then pushes and flows the filtered and purified air 38b through the lower housing unit 22 to the outlet 34. The air purifier 20 thus blows the filtered and purified air 38b back to the ambient environment.

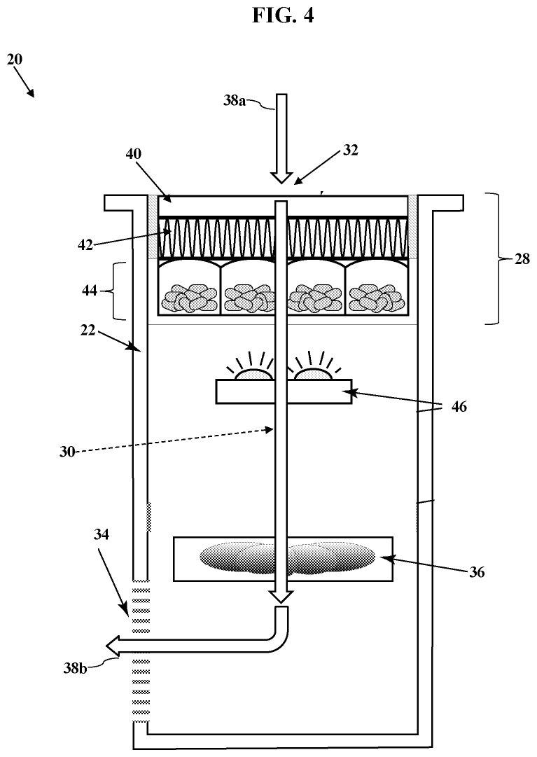

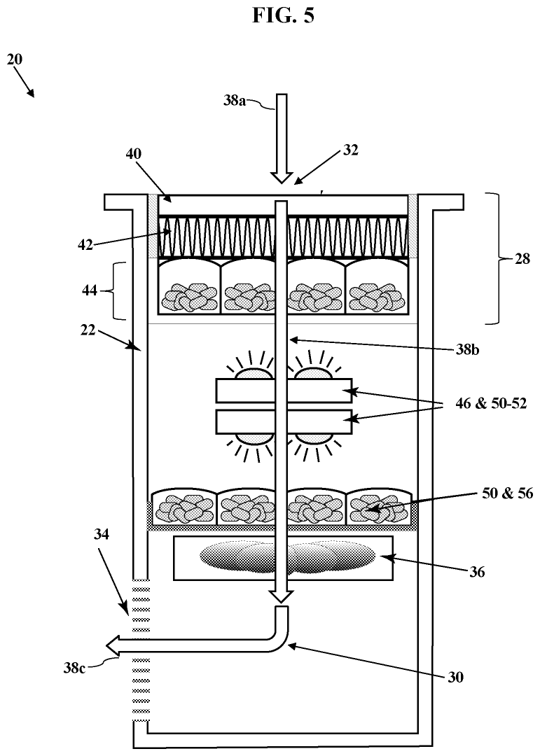

FIGS. 4-5 illustrate some interior, sectional examples of the air purifier 20. The air purifier 20 has the main housing unit 22. The upper photocatalytic purification assembly 28 engages and disengages from the main housing unit 22 (as later paragraphs will explain). When the internal fan assembly 36 receives AC/DC electrical power from a power supply or battery (not shown for simplicity), the internal fan assembly 36 draws the unfiltered, unpurified ambient air 38a through the inlet 32 and into the upper photocatalytic purification assembly 28. The photocatalytic purification assembly 28 continues the airflow path 30 from an upper pre-filter 40, to and through a lower, intermediate or secondary filter 42, and to and through a photocatalytic layer 44. The photocatalytic layer 44 is illuminated by a light source 46. As the internal fan assembly 36 draws the ambient air 38a into and through the upper photocatalytic purification assembly 28, the internal fan assembly 36 draws the ambient air 38a along the internal airflow path 30 into and through the photocatalytic purification assembly 28, thus conducting a double or dual filtration 40 and 42. Moreover, because the photocatalytic layer 44 is exposed to light rays/photons from the light source 46, the photocatalytic layer 44 also performs purification. The photocatalytic layer 44, for example, chemically decomposes pollutants, breaks down volatile organic compounds, eliminates bacteria (such as viruses, fungi, and mold spores) and removes odors. The photocatalytic purification assembly 28 thus produces the filtered and purified air 38b. The internal fan assembly 36 then pushes and flows the filtered and purified air 38b to the outlet 34, and the filtered and purified air 38b exhausts to the ambient environment.

In FIG. 5, two-stage photocatalytic purification is performed. Here the air purifier 20 may have the upper photocatalytic purification assembly 28. The main housing unit 22 may also have a separate, additional lower photocatalytic purification assembly 50. As FIG. 5 illustrates, the light source 46, for example, may be arranged, disposed, or sandwiched between the upper photocatalytic purification assembly 28 and the lower photocatalytic purification assembly 50. The light source 46, for example, may have an upper array 52 of LED light sources that emit light rays/photons upward and into the upper photocatalytic purification assembly 28 and its corresponding photocatalytic layer 44. The light source 46, though, may also have an opposite, lower array 54 of LED light sources that emit light rays/photons downward and into the lower photocatalytic purification assembly 50 and its corresponding lower photocatalytic layer 56. The light source 46, in other words, may project light from both above (i.e., upstream) and from below (i.e., downstream), thus nearly simultaneously illuminating and activating the upper photocatalytic layer 44 and the lower photocatalytic layer 56. So, as the internal fan assembly 36 pulls the ambient air 38a into the air purifier 20, the internal airflow path 30 directs the ambient air 38a into and through the two-stage filtration 40 and 42. The internal airflow path 30 also directs the ambient air 38a into and through the first stage photocatalytic purification assembly 28. The internal airflow path 30 directs the resultant filtered and purified air 38b around and/or though the light source 46 and into the lower photocatalytic purification assembly 50. Because the lower array 54 of LED light sources illuminates the lower photocatalytic layer 56, the lower photocatalytic purification assembly 50 performs second stage purification that further chemically decomposes remaining pollutants, VOCs, bacteria, and odors. The internal fan assembly 36 pushes and flows filtered and double chemically purified air 38c to the outlet 34, and the filtered and double chemically purified air 38c exhausts to the ambient environment.

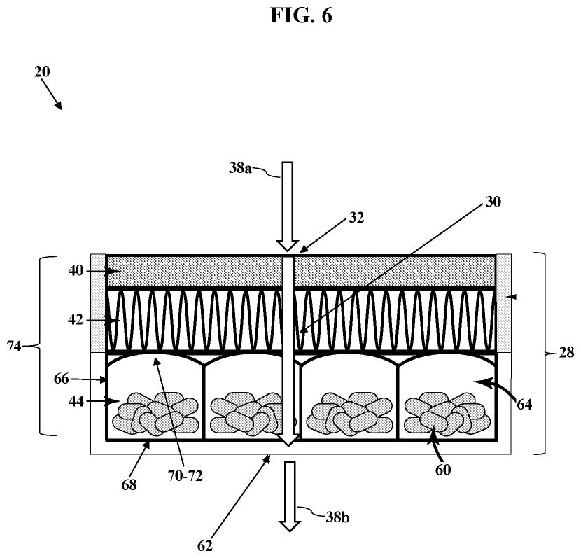

FIG. 6 illustrates more sectional examples of the upper photocatalytic purification assembly 28. The upper photocatalytic purification assembly 28 has the airflow path 30 into and through the upper pre-filter 40, into and through the secondary filter 42, and into and through the photocatalytic layer 44 of photocatalytic material 60. The airflow path 30 exits an outlet 62 in the upper photocatalytic purification assembly 28 and flows into and through the lower, main housing unit 22 (such as previously explained and illustrated). The pre-filter 40 and the secondary filter 42, for example, may be constructed of porous, open-cell polyurethane or polyester. Either or both of the filters 40 and 42, as another example, may be constructed of porous cotton, cellulose (paper), polyester, and other fabrics. Either or both of the filters 40 and 42, as more examples, may be a High-Efficiency Particulate Air (or HEPA) filter or material. The filters 40 and 42 may have whatever design, porous size, composition, and/or material that suits design, cost, and performance objectives.

The airflow path 30 continues down and through the photocatalytic layer 44. The upper photocatalytic purification assembly 28 includes the bed or photocatalytic layer 44 of the photocatalytic material 60. FIG. 6 illustrates the photocatalytic material 60 downstream from the filters 40 and 42 (the photocatalytic material 60, though, may be optionally configured upstream/above the filters 40 and 42). The upper photocatalytic purification assembly 28, for example, may may have a honeycomb-like arrangement of one or many chambers 64. Each chamber 64, for example, may be sized to hold or contain one or more pellets of the photocatalytic material 60. Each chamber 64, for example, may have one or more chamber side walls 66, a chamber bottom floor 68, and a chamber top, ceiling, or roof 70. Each chamber 64 may be machined, molded, printed, or formed from a clear, transparent, or translucent material (such as plastic or other polymer), thus allowing the light rays/photons to pass through the wall/floor/roof 66/68/70 material and into the chamber 64. Moreover, the wall/floor/roof 66/68/70 may have a domed, concave or hemispherical shape 72. As the light rays/photons enter and travel within the chamber 64, the hemispherical shape 72 reduces refraction and promotes reflection. More of the light rays/photons thus remain in the chamber 64 to optimize internal light distribution and to enhance chemical air purification. The filters 40 and 42, along with the chambers 64 retaining the photocatalytic material 60, may be designed, packaged, and sold as a replaceable photocatalytic filter cartridge 74, as later paragraphs will explain.

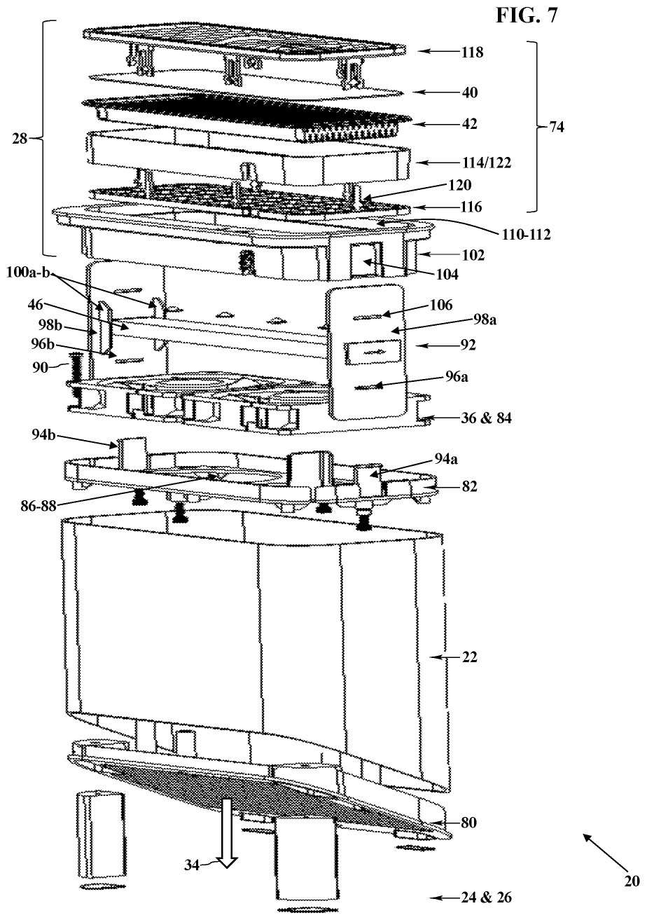

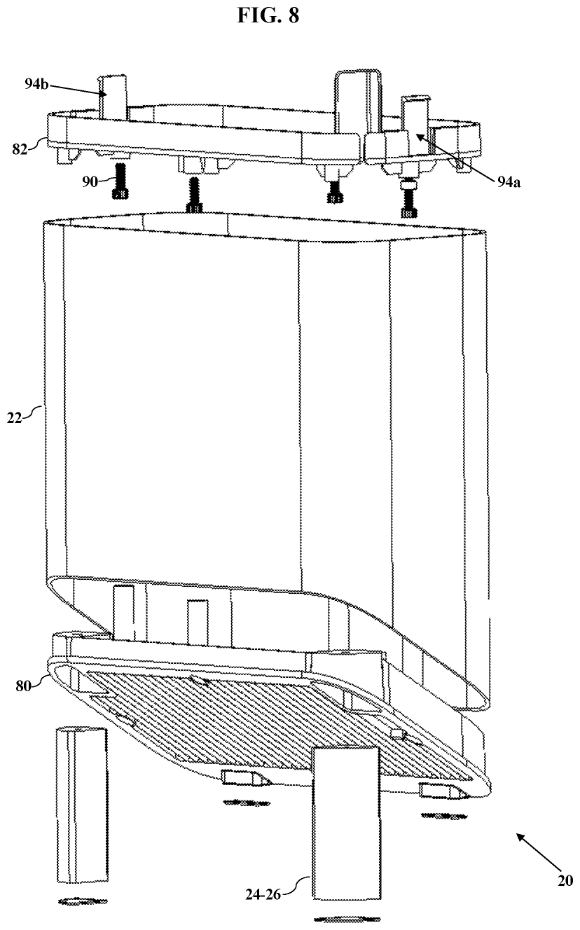

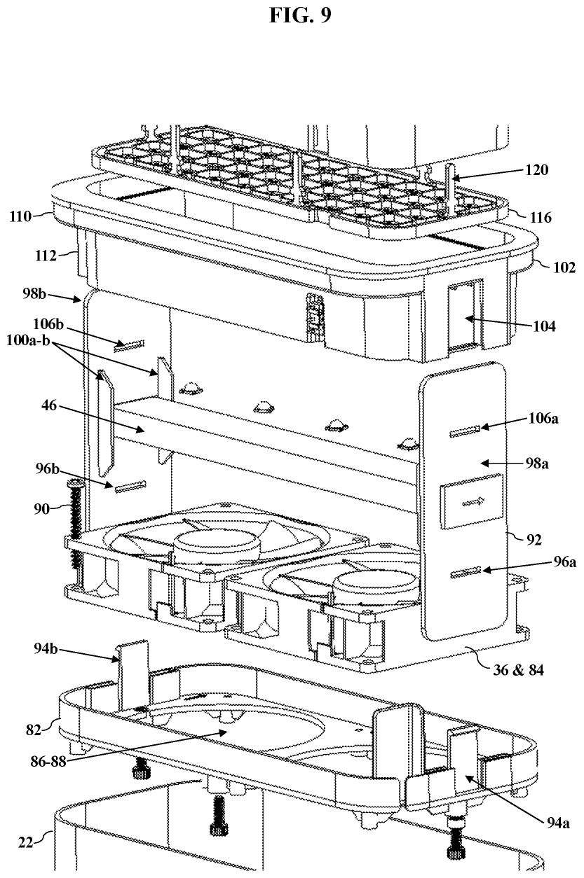

FIGS. 7-10 illustrate exploded assembly examples of the air purifier 20. The air purifier 20 has the lower, main housing unit 22 with the stability legs 24 and feet 26. The main housing unit 22 has a bottom grill 80 providing the air flow outlet 34. While the bottom grill 80 is illustrated as a separate component that inserts and snaps into, and/or mates with, the main housing unit 22, the bottom grill 80 may be integrally molded, machined, printed, or stamped with the main housing unit 22. An internal component harness base 82 inserts proximate an upper portion or region of the main housing unit 22, and the internal component harness base 82 accepts the fan assembly 36. When the fan assembly 36 receives AC or DC electrical power, one or more fan motors 84 spin and propel or move air into and through air flow passages 86 in a structural floor 88 of the internal component harness base 82. Again, while the internal component harness base 82 is illustrated as a separate component that inserts and snaps into, or mates with, the main housing unit 22, the internal component harness base 82 may be integrally molded, machined, printed, or stamped with the main housing unit 22. Moreover, while screws, bolts/nuts, and other mechanical fasteners 90 may attach the fan assembly 36 to the internal component harness base 82 and/or to the upper portion or region of the main housing unit 22, the components 36, 82, 84 may incorporate adhesives, snap tabs, tangs, pins, and more means for fastening the components 36, 82, 84.

The internal component harness base 82 also mates with an upper internal component harness 92. The internal component harness base 82, for example, has upwardly protruding tabs 94a and 94b that respectively align with mating lower slots 96a and 96b in sidewalls 98a and 98b of the upper internal component harness 92. As the internal component harness base 82 and the upper internal component harness 92 are aligned and compressed together, for example, the upwardly protruding tabs 94a and 94b slide along the sidewalls 98a and 98b and into their corresponding lower slots 96a and 96b. Each protruding tab 94a and 94b has a perpendicular tang (not shown) that engages the corresponding lower slot 96a and 96b, thus pliably snapping together and securing the internal component harness base 82 to the upper internal component harness 92. Because the fan assembly 36 is secured to the internal component harness base 82, the fan assembly 36 is thus sandwiched between the internal component harness base 82 and the upper internal component harness 92.

The upper internal component harness 92 may also include the light source 46. The light source 46 (such as the arrays 52 and/or 54 illustrated in FIGS. 4-5). The light source 46 may snap into or secure to the upper internal component harness 92. The sidewalls 98a and 98b, for example, may have inwardly protruding pairs of vertical guide rails 100a and 100b that are spaced/sized to accept the light source 46. The light source 46 may thus be modularly inserted down into the upper internal component harness 92, and the guide rails 100a and 100b maintain an alignment of the light source 46 in relation to the fan assembly 36 and/or in relation to the upper photocatalytic purification assembly 28.

The upper photocatalytic purification assembly 28 mates with the upper internal component harness 92. A bottom receptacle 102, for example, has at least one downwardly protruding tab 104 that aligns with a mating upper slot 106 in the sidewall 98a of the upper internal component harness 92. The bottom receptacle 102, for example, may have a second, or more, downwardly protruding tab that aligns with a corresponding mating upper slot in the sidewall 98b (not shown for simplicity). As the bottom receptacle 102 is aligned and slid into the upper internal component harness 92, the downwardly protruding tab(s) 104 slide along the corresponding sidewall 98 and into the corresponding upper slot 106. Each downwardly protruding tab 104 has a perpendicular tang (not shown) that engages the upper slot 106, thus pliably snapping together and securing the bottom receptacle 102 to the upper internal component harness 92.

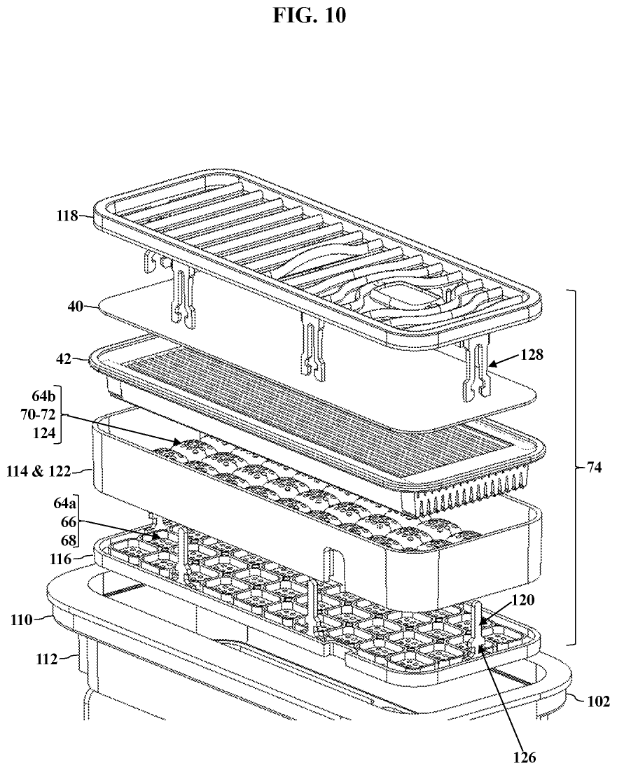

The replaceable photocatalytic filter cartridge 74 inserts into the bottom receptacle 102. The bottom receptacle 102, for example, has an annular rim 110 and a generally rectangular sidewall picture frame 112 that is sized to accept an outer dimension, circumference, and/or shape of the replaceable photocatalytic filter cartridge 74. The replaceable photocatalytic filter cartridge 74, for example, includes a photocatalytic filter bin 114 comprising a bottom tray 116 and a generally rectangular grilled cover 118. The bottom tray 116 may include one or more circumferential, upwardly extending alignment pins 120 that orient, and optionally secure, the bottom tray 116 to the grilled cover 118, thus sandwiching a sidewall 122 defining the photocatalytic filter bin 114 therebetween. As FIG. 10 best shows, the bottom tray 116 may be constructed with the honeycomb-like arrangement of lower half-chambers 64a. Each lower-half chamber 64a, for example, may have the side walls 66 and the perforated chamber floor 68 that retains one or pellets of the photocatalytic material 60 (such as previously explained and illustrated with reference to FIG. 6). The photocatalytic filter bin 114 may have a bin floor 124 that also contains a honeycomb-like arrangement of upper half-chambers 64b. Each upper-half chamber 64b, for example, may have a perforated hemispherical or domed top or roof 70-72 that also retains the one or pellets of the photocatalytic material 60. So, when the replaceable photocatalytic filter cartridge 74 is assembled by aligning and compressing the photocatalytic filter bin 114 between the bottom tray 116 and the grilled cover 118, each the honeycomb-like arrangement of the lower half-chamber 64a aligns with its corresponding honeycomb-like arrangement of the upper half-chamber 64b. Each lower half-chamber 64a thus combines with its corresponding upper half-chamber 64b to form the enclosing chamber 64. Each pin 120 upwardly extending from the bottom tray 116, for example, may have tangs 126 that pliably engage legs 128 downwardly extending from an annular rim of the grilled cover 118. When the fan assembly 36 receives electrical power, the fan assembly 36 flows air into and through each perforated chamber 64 and thus over and through the pellets of the photocatalytic material 60. As air moves through the photocatalytic material 60, each chamber 64 of the photocatalytic material 60 individually chemically scrubs and purifies the air.

The chambers 64 have volumetric sizes. Because the replaceable photocatalytic filter cartridge 74 may include many chambers 64, the chambers 64 may have an equal volumetric size. Some chambers 64, though, may have a different volumetric sizes. As air flows through the air purifier 20, for example, some regions within the photocatalytic filter bin 114 may have greater airflow than other regions. The chambers 64 may thus be volumetrically sized according to the airflow through the chamber 64. Chambers 64 with high air flow, for example, may have a greater volumetric size (e.g., height and/or area) to hold a greater amount of the photocatalytic material 60 (such as more pellets of the photocatalytic material 60). Chambers 64 with low air flow, for example, may have a smaller volumetric size, as fewer pellets may be needed. Chambers 64 that are directly in alignment with the fan assembly 36, for example, may have greater air flow, while chambers 64 peripherally or radially further from a propeller hub or spindle axis may have lesser air flow. The chambers 64 may thus be volumetrically sized from larger to smaller, according to their radial distance from the propeller hub or spindle axis.

The replaceable photocatalytic filter cartridge 74 may also include the filters 40 and 42. The lower, secondary filter 42, for example, may be sized and shaped to inset into an open top formed by the sidewall 122 defining the photocatalytic filter bin 114. The upper pre-filter 40 orients above the lower, secondary filter 42, and the filters 40 and 42 may be fused together as a replaceable, serviceable filter unit. The replaceable photocatalytic filter cartridge 74 may also include the meshed or grilled cover 118 that serves as the intake or inlet 32.

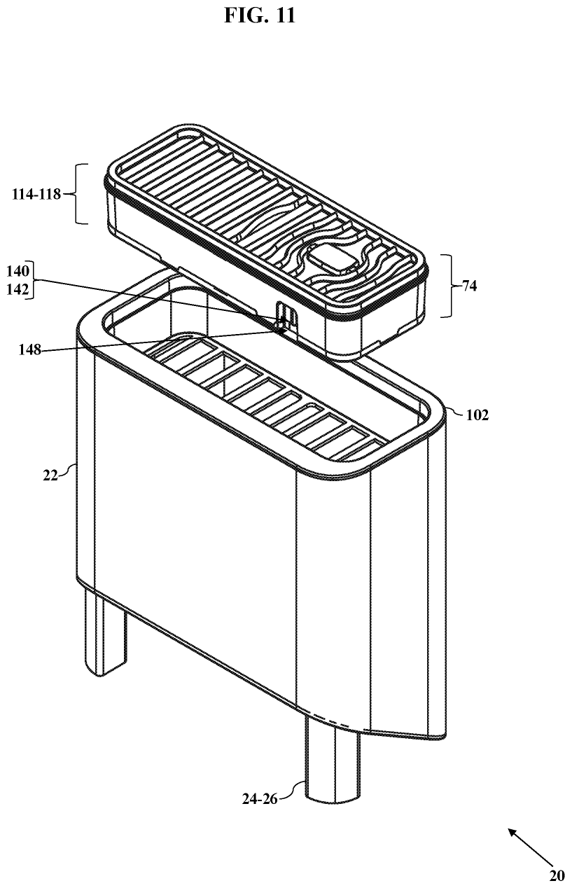

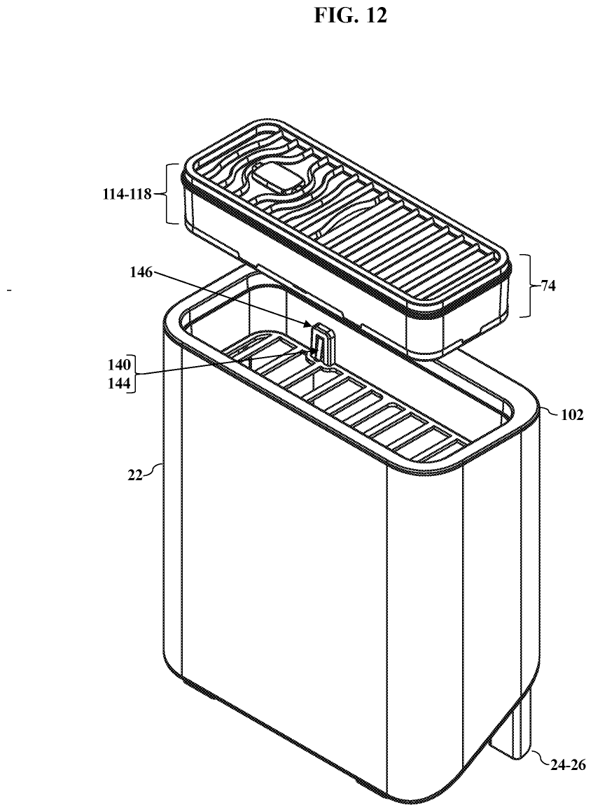

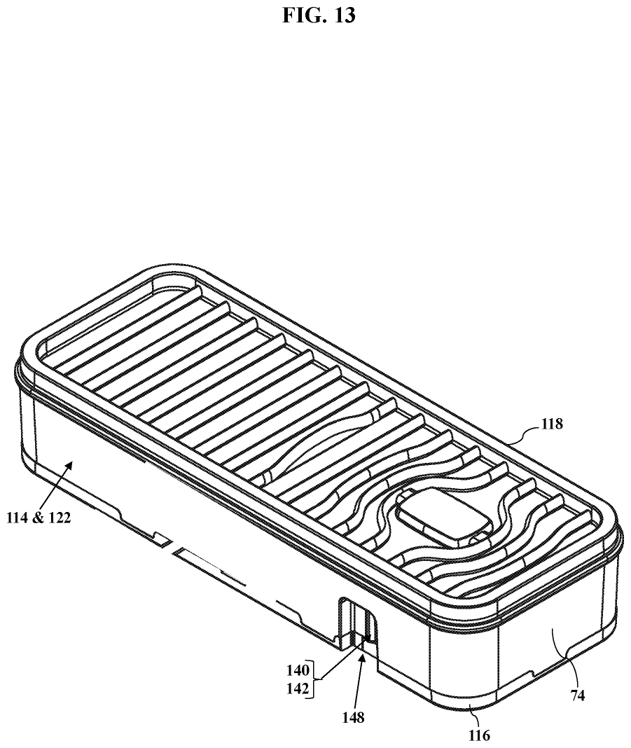

FIGS. 11-13 illustrate examples of electromechanical safety features. The replaceable photocatalytic filter cartridge 74 filters and chemically treats the ambient air 38 (such as previously explained). If the photocatalytic filter cartridge 74 is not properly inserted, aligned, or seated within the bottom receptacle 102 and/or the lower, main housing unit 22, then the air purifier 20 may draw in ambient air 38 without complete filtration nor purification. The fan assembly 36, for example, could suck ambient air 38 from an improper seal between the photocatalytic filter cartridge 74 and the bottom receptacle 102. Indeed, if the photocatalytic filter cartridge 74 was removed from the bottom receptacle 102, there could be a possibility of user injury due to the spinning fan assembly 36 and/or electrical hazards. The air purifier 20 may thus include an electromechanical presence detector 140 for the photocatalytic filter cartridge 74. FIGS. 11-12, for example, illustrate, respectively, isometric front and rear views of the air purifier 20, while FIG. 13 illustrates an isometric view of the photocatalytic filter cartridge 74. As FIGS. 11 and 13 best illustrate, the photocatalytic filter cartridge 74 may include a downwardly protruding electrical switch engager 142, while FIG. 12 illustrates an electrical switch 144. When the photocatalytic filter cartridge 74 is aligned and fully inserted into the bottom receptacle 102, the downwardly protruding switch engager 142 contacts and electrically closes the electrical switch 144. When the electrical switch 144 is closed, current and voltage may flow through the electrical switch 144, thus allowing electrical power to flow from a power supply to the fan assembly 36 and/or to the light source 46 (all not shown for simplicity). If, however, the photocatalytic filter cartridge 74 is not accurately aligned with, and/or nor fully inserted into, the bottom receptacle 102, the downwardly protruding switch engager 142 does not contact/close the electrical switch 144. The electrical switch 144 remains open, thus preventing electrical power to the power supply and/or to the fan assembly 36 and/or to the light source 46. The electromechanical presence detector 140 must therefore sense the proper presence of the photocatalytic filter cartridge 74 to permit electrical operation.

The electromechanical presence detector 140 may also include additional alignment features. The bottom receptacle 102, as previously explained, has the sidewall picture frame 112 defining an inner socket that is sized and shaped to accept the outer dimension, circumference, and/or shape of the photocatalytic filter cartridge 74. Moreover, an interior side of the sidewall picture frame 112 may include a shelf member 146 that inwardly extends or protrudes from the interior side of the sidewall picture frame 112 defining the open, inner socket. The shelf member 146 may incorporate the electrical switch 144. The photocatalytic filter bin 114 may have a corresponding receptacle 148 that is sized and shaped to accept the shelf member 146. The receptacle 148, for example, may be inwardly formed, machined, printed, or broached from the bottom tray 116 and/or the sidewall 122 defining the photocatalytic filter bin 114. Because the receptacle 148 and the shelf member 146 are sized and shaped for alignment, the photocatalytic filter cartridge 74 must be correctly aligned and fully inserted to ensure the downwardly protruding switch engager 142 contacts and electrically closes the electrical switch 144. If, however, the shelf member 146 is not accurately aligned with, and/or nor fully inserted into, the receptacle 148, then the downwardly protruding switch engager 142 does not close the electrical switch 144. Again, then, the electromechanical presence detector 140 must sense the presence of the photocatalytic filter cartridge 74 to permit electrical operation. The electromechanical presence detector 140 is thus a circuit-breaking authentication interlock that physically disables electrical power to the fan assembly 36 and/or to the light source 46 without proper cartridge seating.

The electromechanical presence detector 140 may thus be an authentication and safety interlock system. The photocatalytic filter cartridge 74 incorporates the physical authentication system that serves dual purposes. For example, the electromechanical presence detector 140 ensures proper cartridge seating before device operation. The electromechanical presence detector 140 may also prevent operation of the light source 46 without the photocatalytic filter cartridge 74 in place (e.g., a safety function). The electromechanical presence detector 140 authenticates, for example, via the notch or receptacle 148 in the photocatalytic filter cartridge 74 (such as front-right position) containing the downwardly protruding electrical switch engager 142. The corresponding switch 144 in the receptacle 148 is positioned to be actuated by the electrical switch engager 142. The switch 144, for example, may be wired in series with a main power circuit (not shown for simplicity). The mirrored notch or receptacle 148, inset in the photocatalytic filter cartridge 74, accommodates the protrusion geometry when assembled. The below table further explains the design characteristics.

| Feature | Description |
|---|---|
| Circuit-Breaking | Physical interruption of power circuit (not signal-based). |
| | Without switch actuation, NO powered components operate |
| | (fan AND LEDs both disabled). |
| Orientation Keying | Asymmetric protrusion location (front-right only) prevents 90- |
| | degree or 180-degree misorientation of cartridge. |
| Seating Verification | Dimensional tolerances on protrusion geometry ensure switch |
| | actuation only occurs when cartridge is fully and correctly |
| | seated. |
| Failsafe Design | System fails safe (device inoperable) rather than failing |
| | dangerous (device operates without filter). |

The photocatalytic filter bin 114, replaceable and serviceable as the cartridge 74, may further include orientation control systems. The cartridge 74, for example, incorporates multiple orientation control features, such as further described in the below table.

| Junction | Method | Type |
|---|---|---|
| Cartridge to Device | Asymmetric authentication | Mechanical |
| | protrusion position | forcing |
| Bottom to Middle | Orientation post on bottom | Mechanical |
| Housing | housing engages socket in | forcing |
| | middle housing | |
| Top to Middle Housing | Logo integrated into grill | Visual |
| | indicates correct orientation | indicator |

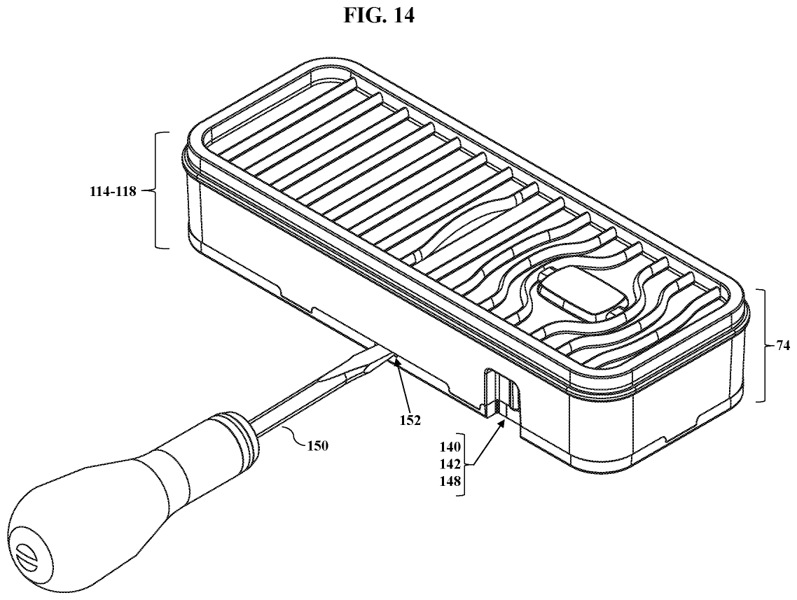

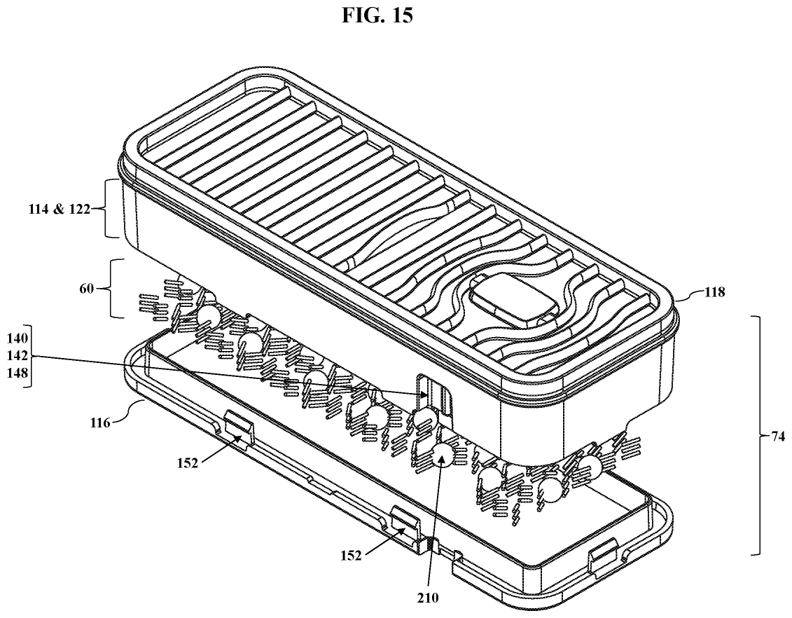

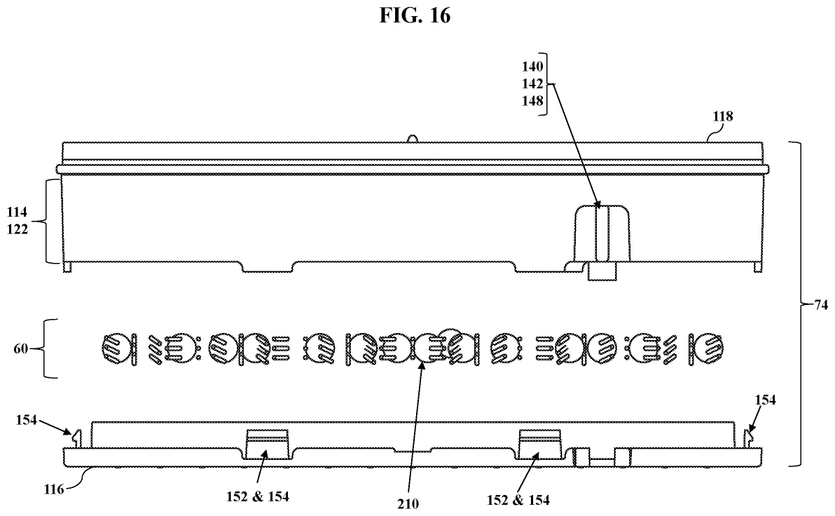

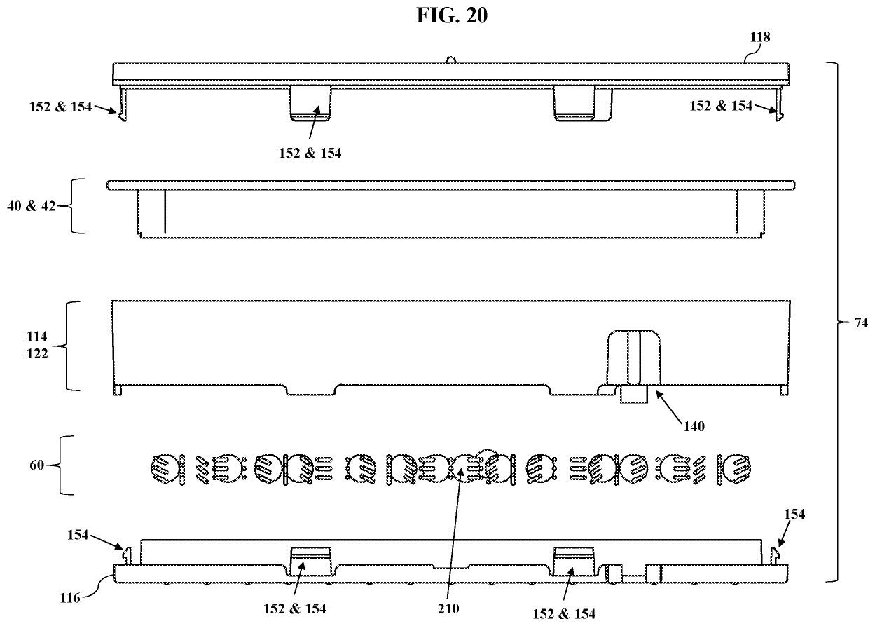

FIGS. 14-22 illustrate examples of servicing the photocatalytic filter cartridge 74. The photocatalytic filter cartridge 74 may be disassembled, and reassembled, for replacing the filters 40 and 42. The photocatalytic filter cartridge 74 may also be disassembled, and reassembled, for washing or replacing the photocatalytic material 60. In FIG. 14, for example, the tip of a flathead screwdriver 150 may be inserted into one or more disassembly notches 152. A pushing and prying or twisting movement of the tip, for example, separates tanged tabs 154 in the bottom tray 116 from engaging mates in the photocatalytic filter bin 114 (such as previously explained with reference to FIGS. 7 & 10). FIGS. 15-16 thus illustrate a disassembled, exploded view of the photocatalytic filter cartridge 74. Once a user or technician exposes the photocatalytic material 60, the user or technician may remove and clean or replenish the photocatalytic material 60. The user or technician may may then reassemble the photocatalytic filter bin 114 by matching and pressing the upwardly extending tanged tabs 154 in the bottom tray 116 into their corresponding securement features in the sidewall 122 defining the photocatalytic filter bin 114.

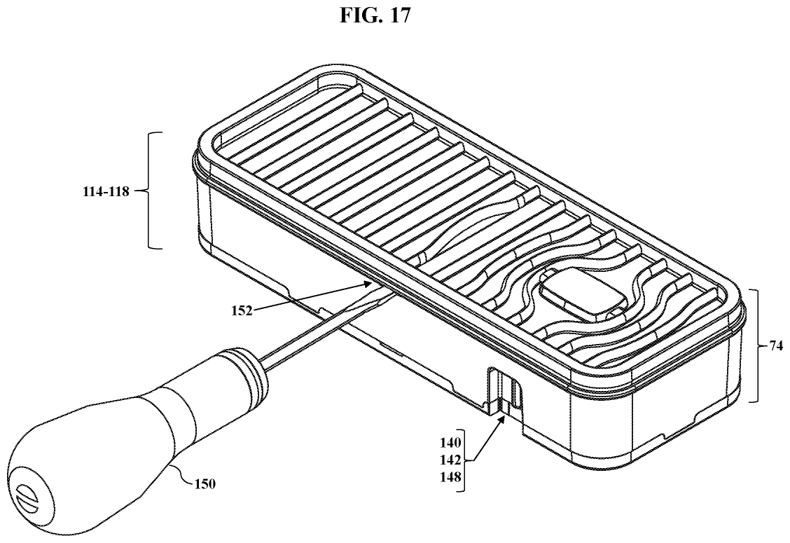

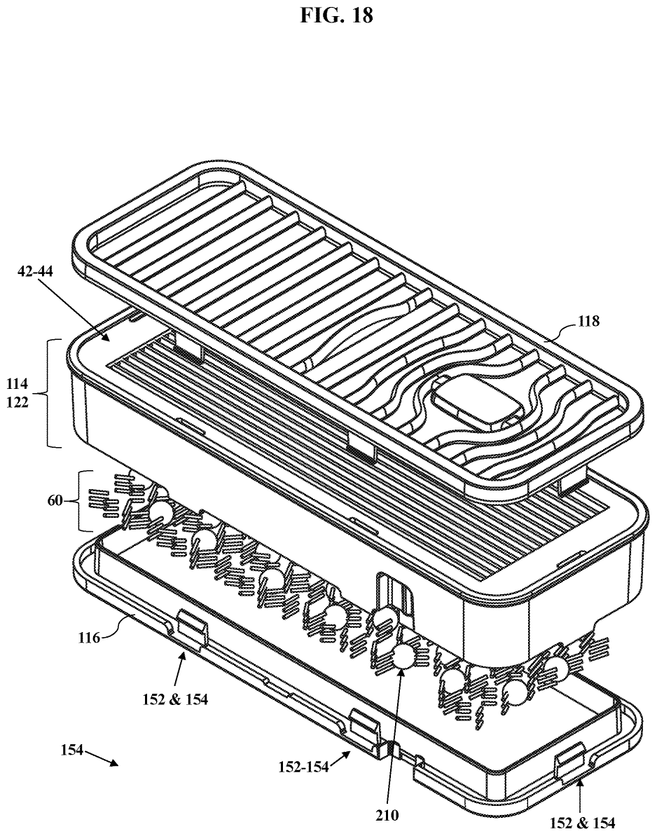

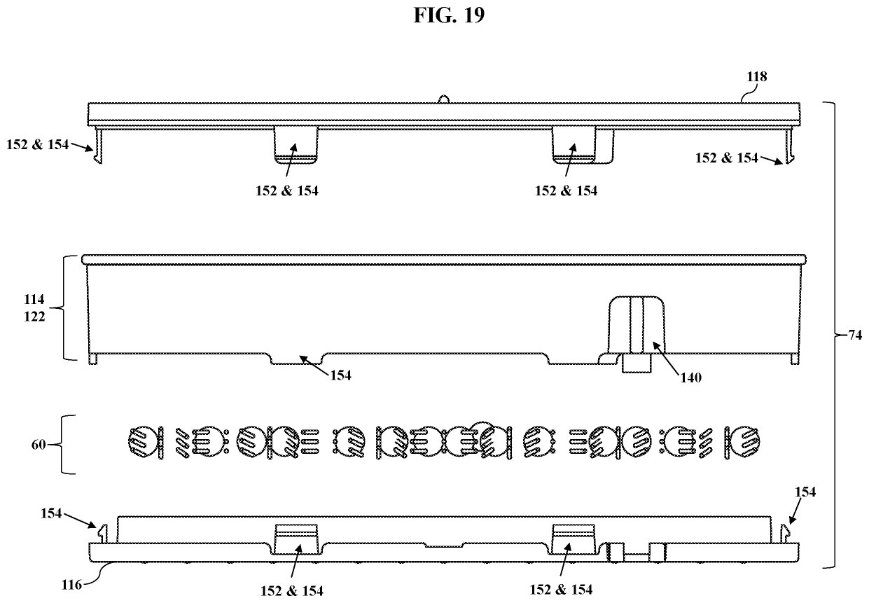

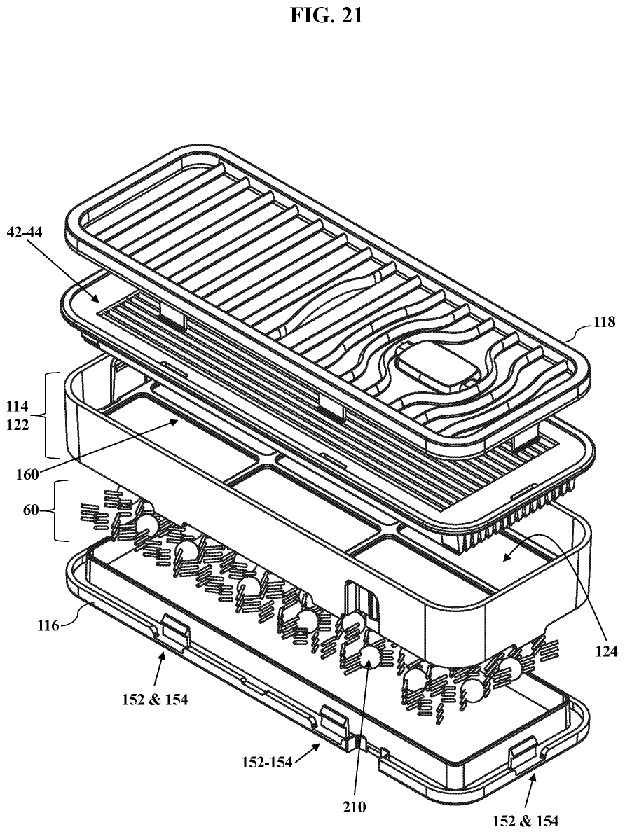

FIGS. 17-21 illustrate more examples of additional servicing. The photocatalytic material 60 may be exposed by removing the bottom tray 116 from engagement with the sidewall 122 defining the photocatalytic filter bin 114 (such as previously explained). As FIGS. 17-18 illustrate, the grilled or meshed cover 118 may also be removed from engagement with the sidewall 122 defining the photocatalytic filter bin 114 (such as by inserting the flathead tip of the screwdriver 150 into more disassembly notches 152 and moving or pressing tanged tabs 154 downwardly extending from the grilled or meshed cover 118). Once the user or service technician separates the grilled or meshed cover 118, the filters 40 and 42 and the photocatalytic material 60 are exposed for servicing. FIG. 19 thus illustrates the exploded and disassembled three-piece photocatalytic filter cartridge 74 (containing the photocatalytic material 60). The photocatalytic material 60 may thus be sold and easily replaced for periodic usage. The photocatalytic filter cartridge 74 may thus comprise a modular assembly with tool-release mechanisms designed for repeated factory disassembly and reassembly. The filters 40 and 42 and the photocatalytic material 60, though, may be individually marketed and sold for replacement. Once the replaceable components 40-42 & 60 are installed, the grilled or meshed cover 118 may be re-attached with the sidewall 122 defining the photocatalytic filter bin 114 (such as by aligning and inserting the tanged tabs 154). As FIG. 21 best illustrates, the bin floor 124 in the photocatalytic filter bin 114 may have an optional or additional ribbed construction that separates the lower, secondary filter 42 from the photocatalytic material 60. The bin floor 124 may have longitudinal and transverse crossmembers 160 that support the filters 40 & 42. The crossmembers 160 define airflow passages from the filters 40-42 to the photocatalytic material 60.

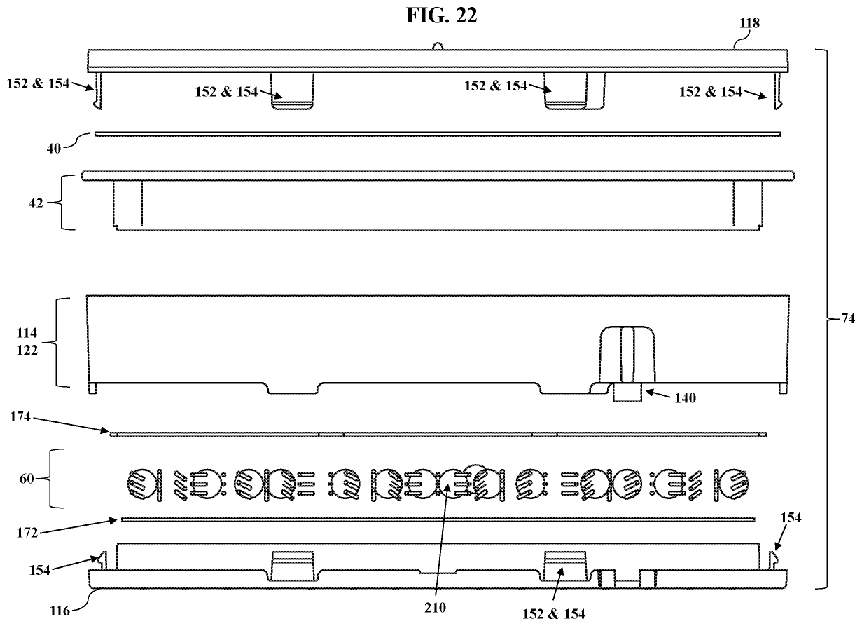

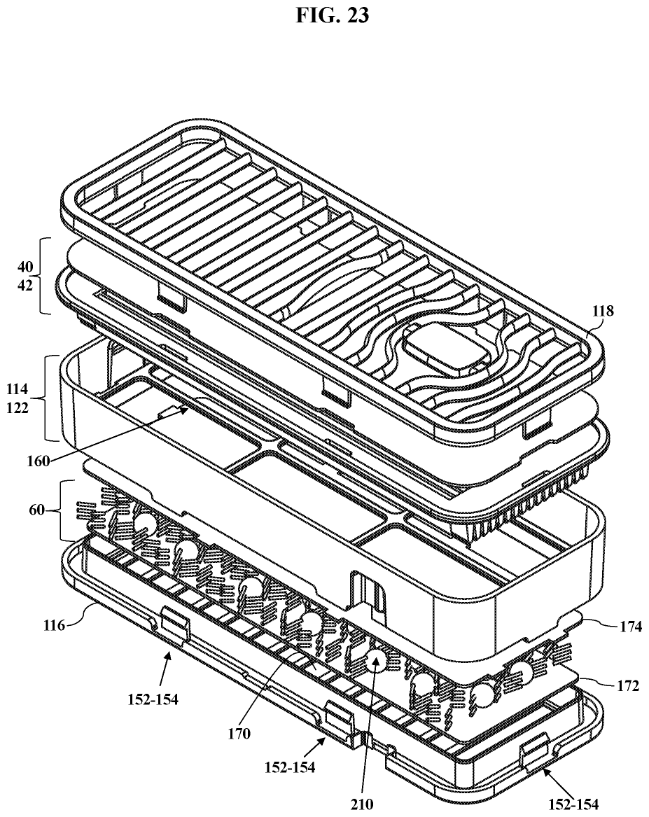

FIGS. 22-23 illustrate more examples of more photocatalytic features. The photocatalytic filter cartridge 74 contains the filters 40-42 and the contains the photocatalytic material 60. The bottom tray 116, for example, may have a ventilated, perforated, or slotted floor 170 that promotes air passage out of the photocatalytic material 60. Depending on the size(s) of the air passages, a lower mesh screen or lattice sheet 172 may be inserted between the ventilated floor 170 and the photocatalytic material 60. The mesh screen or lattice sheet 172 still flows air out of the photocatalytic material 60, but the screen/sheet 172 prevents the photocatalytic material 60 from falling through the bottom tray 116. The photocatalytic filter bin 114, as more examples, may have the ventilated, perforated, or slotted bin floor 124 having the crossmembers 160 that define airflow passages into the photocatalytic material 60. Again, depending on the size(s) of the airflow passages, an upper mesh screen or lattice sheet 174 may be inserted between the floor/crossmembers 124/160 and the photocatalytic material 60. The photocatalytic filter bin 114, for example, may contain the photocatalytic material 60 in 6 mm deep bed. The photocatalytic filter bin 114 may thus hold pellets, beads, or capsules of the visible-light-activated photocatalytic material 60 on a diatomaceous earth substrate. The pellets, for example, may be approximately 1 mm width, oblong shape, and/or variable length. The dual mesh screen 172-174 construction, for example, creates a photocatalyst sandwich. The mesh screens 172-174 permit airflow while retaining pellets regardless of the cartridge's 74 orientation.

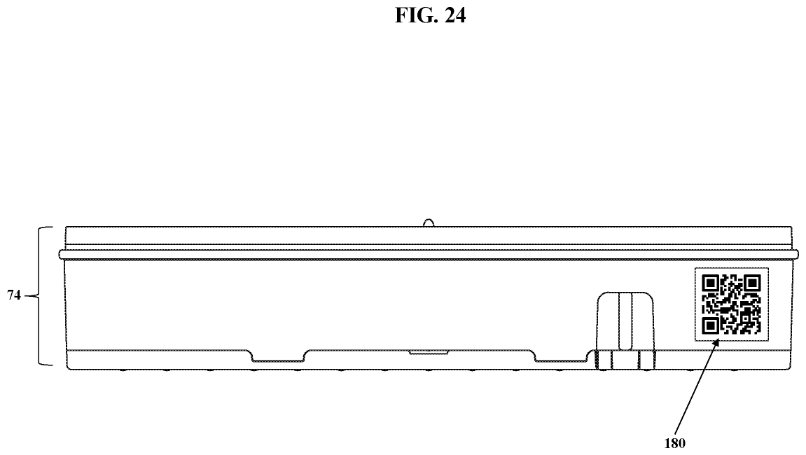

FIG. 24 illustrates examples of a QR-based lifecycle tracking system. The photocatalytic filter cartridge 74 may include a molded, embossed, or adhesive QR code 180. The QR code 180 may be unique identifier mechanism for logging and tracking lifecycle usage of the photocatalytic filter cartridge 74. A smartphone camera, for example, may be used to scan the QR code 180. The smartphone may then establish communication with a cloud server or service that tracks and monitors an installation or service date of the photocatalytic filter cartridge 74. When the QR code 180 reveals a stale or expired time since installation or service, then the cloud server or service may initiate text messages, emails, and other reminder notifications. The QR code 180 may thus reveal the cartridge's refurbishment history and housing retirement decisions. Moreover, the QR code 180 may be affixed to the exterior side of the photocatalytic filter cartridge 74. This exterior side placement is advantageous, as the structural components 114-118 are not ordinary replaced during refurbishment for maximum persistence. The structural components 114-118, of course, may be replaced due to damage that is beyond repair, so a new QR code 180 may be assigned to the customer's or purifier's account and service records. Regardless, the QR code 180 may uniquely track the structural asset, not the consumable components 40-42 & 60. The exterior side placement is also protected from wear while remaining accessible for scanning. Moreover, the same component carries the authentication components 140-142 & 148, creating a unified “identity” element. The below table explains more design features.

| Function | Description |
|---|---|
| Unit Identification | Each cartridge has a unique identifier linked to a central |
| | database record |
| Refurbishment Tracking | Database records each return/refurbishment event with |
| | timestamp and actions taken |
| Lifecycle Management | Cycle count tracking enables housing retirement when |
| | predetermined threshold is reached |
| Quality Control | Batch tracking enables identification of manufacturing issues; |
| | fraud detection for counterfeit cartridges |
| Consumer Engagement | App-based feature allows consumers to view map of previous |
| | locations where their cartridge has been deployed |

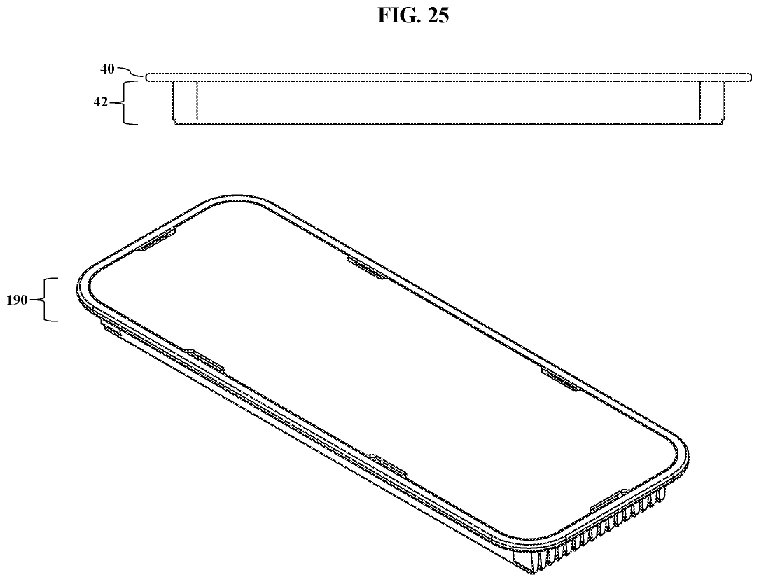

FIG. 25 illustrates examples of a fused filter component 190. Here the pre-filter 40 and the secondary filter 42 may combined as the single, fused filter component 190. The fused filter component 190 may thus be marketed and sold as a single-piece replaceable and serviceable filter cartridge. The particulate filtration stages (such as the pre-filter 40 and the secondary HEPA filter 42) may be combined into the single fused filter component 190 that is replaced as one component. This design choice simplifies manufacturing (one unit to install), simplifies refurbishment (one unit to remove/replace), eliminates reassembly errors (no risk of incorrect filter stacking), and creates a defined consumable SKU for recurring revenue.

The photocatalytic filter cartridge 74 thus has the three-part modular housing architecture 114-118. The photocatalytic filter cartridge 74, for example, has three distinct housing components: top (such as the grilled or meshed cover 118), middle (such as the generally rectangular photocatalytic filter bin 114), and the bottom tray 116. Each section serves a specific function and may contain different filtering and/or purifying elements with different replacement frequencies. This architecture enables selective component replacement during refurbishment, maximizing material recovery and minimizing waste. The below table provides additional component description.

| Component | Contents | Refurbishment Action |
|---|---|---|
| Cover | Fused pre-filter/HEPA unit | Replace fused filter unit every cycle; |
| | (single replaceable element); | clean and reuse housing |
| | ventilation grill with integrated | |
| | logo for visual orientation | |
| Bin | Structural frame; airflow path; | Clean and reuse; QR scan for |
| | authentication protrusion; upper | lifecycle tracking |
| | mesh screen (attached to bottom | |
| | face); QR code identifier (side) | |
| Bottom Tray | Photocatalytic media chamber | Inspect pellets; replace only if |
| | containing DE pellets; upper and | contaminated; clean and reuse |
| | lower mesh screens (pellet | housing |
| | sandwich); orientation post | |

The tool-release mechanisms aid factory servicing. The grilled or meshed cover 118, the photocatalytic filter bin 114, and the bottom tray 116 may each include the housing junction features 150-152 that provide a secure but releasable connection system. For example, the multiple (e.g., 6) molded tabs 152 on each housing section snap into their corresponding slots when assembled. The screwdriver-head sized notches 150 on both sides of each component 114-118, for example (such as four notches 150 total: two for cover-to-bin junction, two for tray-to-bin junction). Tool insertion and rotation releases the tabs 152, allowing non-destructive separation. Dual-side release points enable balanced force distribution, preventing binding and extending housing lifecycle through multiple refurbishment cycles. This mechanism may be deliberately designed for factory disassembly, not consumer serviceability. The tool requirement and small notch size discourage consumer tampering while enabling efficient refurbishment by trained technicians.

The air purifier 20 may have many added features. For example, while the light source 46 may be activated to emit any portion of the electromagnetic spectrum, the light source 46 may emit visible-light (≈405 nm). While other electromagnetic frequencies may have burdensome and costly regulatory requirements, the air purifier 20 may use visible light purification with little or no regulatory concerns. The air purifier 20 may thus be marketed and sold as a consumer safe product that may avoid UV classifications, but the air purifier 20 still achieves odor/VOC reduction comparable to UV systems. The photocatalytic material 60 (e.g., pelletized or granular) may be easily replaced by consumers and technicians. The air purifier 20 may incorporate the pre-filter 40 and the secondary filter 42 as a single, replaceable filter unit. The photocatalytic material 60 in the photocatalytic filter bin 114 may also be marketed and sold as a single, replaceable photocatalytic packet or cartridge.

The air purifier 20 may include optical enhancements. The lower and upper half-chambers 64a-b, for example, may include optical chamber geometry that is designed for photon economy (such as reduced usage and energy consumption by the light source 46 in a small home air purifier 20). The upper half-chambers 64b, for example, may have the domed or hemispherical roof 70-72 that at least partially reduces refraction and that reflects the light photons/waves back into the photocatalytic material 60. The chambers 64, though, may incorporate other features, such as transmissive walls, curved/faceted or reflective features that increase light dose through the photocatalytic pellet bed.

As FIGS. 15-22 also illustrate, the air purifier 20 may include light-distributing, optically transparent elements scattered within the photocatalytic material 60. To improve photocatalytic efficiency, clear glass or plastic transparent spheres 210 (5-6 mm diameter), for example, may be interspersed and mixed in with the photocatalytic material 60. These optically transparent spheres 210 act as light-distributing elements that scatter and redirect light deeper into the photocatalytic material 60. The optically transparent spheres 210, in other words, reflect and re-emit incident light. Each optically transparent sphere 210 receives light from the light source 46, and the some of the light will reflect off the spherical surface and illuminate neighboring or proximate photocatalytic beads, pellets, capsules, or other shapes. Some of the light, though, will refract into the optically transparent sphere 210, illuminate an interior of the optically transparent sphere 210, and refract out of the optically transparent sphere 210. This internal reflection, refraction, and re-emission of light from all sphere surfaces (including sides and bottom) will illuminate the neighboring photocatalytic elements that would otherwise be shadowed. Each optically transparent sphere 210 effectively becomes a secondary light source embedded within the photocatalytic media bed. In a 6 mm deep chamber filled with 1 mm-width photocatalytic pellets, for example, light attenuation occurs rapidly due to pellet-to-pellet shadowing. Each optically transparent sphere 210 addresses this light attenuation by receiving light from the light source, internally reflecting and refracting the light within the optically transparent sphere 210, re-emission of light from all internal sphere surfaces (including sides and bottom), and illumination of proximate pellets that would otherwise be shadowed. The light-distributing optically transparent elements, though, may have other physical shapes, such as prisms, cylindrical rods, cubes, tablets, flakes, mirrors, or other 2D/3D shapes to scatter and redirect light deeper into the photocatalytic material 60.

The air purifier 20 may include electronic sensors. A photoelectric cell, for example, may be configured within the air purifier 20 to sense the light exiting the photocatalytic material 60. The photoelectric cell, for example, may generate a resistance value and/or an output signal in relation to the light entering the photoelectric cell. The air purifier 20 may include a hardware processor, ASIC, memory, or other electronic circuit that compares the resistance value and/or the output signal to a reference value. If the resistance value and/or the output signal is greater than or equal to the reference value, for example, then the photocatalytic material 60 is optically transmitting light, and/or the light source 46 is adequately emitting light. The air purifier 20, in other words, may be normally functioning. If, however, the resistance value and/or the output signal is less than the reference value, then air purifier 20 may be abnormally functioning. The photocatalytic material 60, for example, may be clogged with particulate matter that passes through the filters 40 and 42 and causes reduced optically transmittance. The photocatalytic material 60, as another example, may be chemically exhausted and no longer effective. The light source 46, as another example, may have dimmed or inadequate output. Whatever the cause, though, the electronic circuit may activate a replacement/service warning light and/or initiate BLUETOOTH®, WIFI®, cellular alerts/messages via a network interface and/or radio transmitter. Moreover, the electronic circuit may be programmed to adjust and reduce the voltage applied to the fan assembly 36, thus reducing its rotational speed and reducing the airflow. The electronic circuit may be programmed to adjust and reduce the voltage applied to the light source 46, thus reducing its light output. The electronic circuit may thus reduce the performance of the air purifier 20 until the root cause problem is diagnosed and resolved.

The air purifier 20 may have many additional features. When the air purifier 20 has the light source 46 installed between the upper photocatalytic purification assembly 28 and the lower photocatalytic purification assembly 50 (as illustrated with reference to FIG. 5), the light source 46 may have dual-band or multi-wavelength configurations to activate multiple types of photocatalysts. The upper array 52 of LED light sources, for example, may emit light rays/photons upward and into the upper photocatalytic purification assembly 28 at one portion or band within the electromagnetic spectrum. The opposite, lower array 54 of LED light sources, though, may emit light rays/photons downward and into the lower photocatalytic purification assembly 50 at a different portion or band within the electromagnetic spectrum. The multiple types of photocatalysts may thus have different photocatalytic properties and/or chemical reactions when activated at their corresponding frequencies. The multiple types of photocatalysts, and the upper array 52 and lower array 54 of LED light sources, may thus be chosen for different chemical purifications.

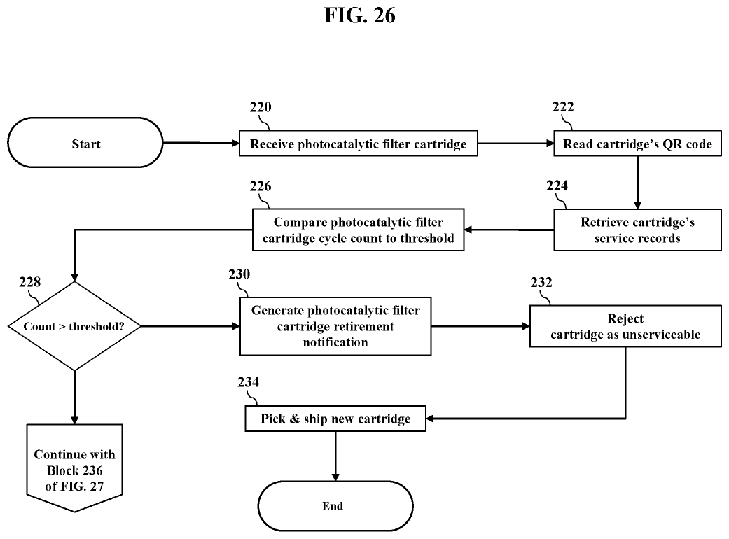

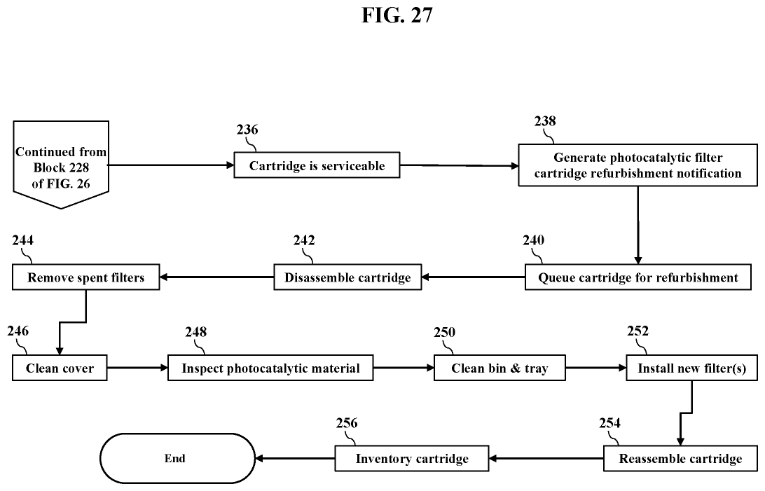

FIGS. 26-27 illustrate examples of methods and operations for centralized, factory refurbishment of the air purifier 20 and/or the photocatalytic filter cartridge 74. The modular cartridge architecture 114-118 enables a centralized selective component replacement process. This method, for example, represents a factory-based refurbishment system. A consumer user, for example, ships or delivers the used photocatalytic filter cartridge 74 to central refurbishment facility. When the used photocatalytic filter cartridge 74 is received (Block 220) at the central refurbishment facility, technicians or sorting machinery scan the QR code 180 on the used photocatalytic filter cartridge 74 (Block 222). A computerized inventory database system uses the QR code 180 as a unique identifying query parameter to retrieve service records associated with the used photocatalytic filter cartridge 74 (Block 224). A computer server's CPU/GPU and memory device, for example, perform a database lookup and retrieves a service history associated with the used photocatalytic filter cartridge 74. The service technician or the computer server, as another example, may be programmed to compare a photocatalytic filter cartridge cycle count against a predetermined threshold value (Block 226). If the photocatalytic filter cartridge cycle count exceeds the threshold value (Block 228), then the service technician or the computer server determines that the used photocatalytic filter cartridge 74 has exceeded its lifecycle and the computer server generates and logs a photocatalytic filter cartridge retirement notification (Block 230). The photocatalytic filter cartridge retirement notification, for example, may be a text message, email, or other notification that is sent to notification addresses. The photocatalytic filter cartridge retirement notification, for example, may be sent to receiving personnel and/or to a receiving department's sorting machine. The photocatalytic filter cartridge retirement notification may thus cause or instruct the receiving personnel and/or the sorting machine to reject the spent photocatalytic filter cartridge 74 as unserviceable (Block 232). The service technician or the computer server, for example, may instruct the sorting machine to pick a brand new photocatalytic filter cartridge 74, scan and record its corresponding QR code 180, and ship the brand new photocatalytic filter cartridge 74 back to the customer (Block 234).

The flowchart continues with FIG. 27. The used photocatalytic filter cartridge 74, though, may be serviceable. For example, if the photocatalytic filter cartridge cycle count equals or is less than the threshold value (see Block 228 of FIG. 26), then the computer server determines that the spent photocatalytic filter cartridge 74 is serviceable (Block 236). The computer server, for example, is programmed to perform operations that generate a photocatalytic filter cartridge refurbishment notification (Block 238). The photocatalytic filter cartridge refurbishment notification, for example, may be a text message, email, or other notification that is sent to notification addresses. The photocatalytic filter cartridge refurbishment notification, for example, may be sent to the receiving personnel and/or to the receiving department's sorting machine. The photocatalytic filter cartridge refurbishment notification may thus cause or instruct the receiving personnel and/or the sorting machine to queue the spent photocatalytic filter cartridge 74 for human refurbishment (Block 240).

The service technician refurbishes the photocatalytic filter cartridge 74. The service technician, for example, disassembles the photocatalytic filter cartridge 74 (Block 242). The technician, for example, inserts the tool screwdriver 150 and releases the grilled or meshed cover 118 from engagement with the photocatalytic filter bin 114 (such as explained with reference to FIGS. 14-23). The service technician may then remove and dispose of the spent/clogged filters 40 and 42 (Block 244). The service technician may also inspect and clean the grilled or meshed cover 118 and then set aside for reuse (Block 246). The service technician may also insert the screwdriver 150 and release the bottom tray 116 from the photocatalytic filter bin 114 (such as explained with reference to FIGS. 14-23). The service technician visually inspects and, if needed, replaces the photocatalytic material 60 (Block 248). If the photocatalytic material 60 is clean and shows little contamination (dust coating, discoloration), for example, then the service technician may retain the photocatalytic material 60 in place without replacement. If, however, the photocatalytic material 60 exhibits or shows unacceptable contamination, then the service technician removes and replaces with fresh photocatalytic material 60. The service technician may also inspect and clean accessible areas of the photocatalytic filter bin 114 and the bottom tray 116 (Block 250). The service technician installs new, fused pre-filter/HEPA unit (Block 252) and reassembles the photocatalytic filter cartridge 74 (Block 254). The service technician and/or the computer server logs/records a refurbishment event in the computerized inventory database system (such as increment cycle count, log actions taken in association with QR code 180). The service technician may then return the refurbished photocatalytic filter cartridge 74 to inventory for redistribution to consumers (Block 256).

As used herein, the singular forms “a,” “an,” and “the” are intended to include the plural forms as well, unless expressly stated otherwise. It will be further understood that the terms “includes,” “comprises,” “including,” and/or “comprising,” when used in this Specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. Furthermore, “connected” or “coupled” as used herein may include wirelessly connected or coupled. As used herein, the term “and/or” includes any and all combinations of one or more of the associated listed items.

It will also be understood that, although the terms first, second, and so on, may be used herein to describe various elements, these elements should not be limited by these terms. These terms are only used to distinguish one element from another. For example, a first computer or container could be termed a second computer or container and, similarly, a second device could be termed a first device without departing from the teachings of the disclosure.

1. An apparatus for filtering and purifying air, comprising:
a photocatalytic filter cartridge having an interior airflow path from an air inlet at a cover secured to a photocatalytic filter bin, and the interior airflow path continuing through the photocatalytic filter bin to an air outlet in a bottom tray secured to the photocatalytic filter bin;
a pre-filter within in the photocatalytic filter bin downstream the interior airflow path from the air inlet cover;
a secondary filter within the photocatalytic filter bin downstream the interior airflow path from the pre-filter; and
a photocatalytic material downstream the interior airflow path from the secondary filter and retained in the photocatalytic filter bin by the bottom tray;
wherein the photocatalytic filter cartridge filters and purifies air flowing along the interior airflow path from the air inlet at the cover to the air outlet in the bottom tray.

2. The apparatus of claim 1, wherein the photocatalytic filter cartridge further comprises a hemispherical chamber containing the photocatalytic material.

3. The apparatus of claim 1, further comprising a bin floor in the photocatalytic filter bin having an upper array of hemispherical half chambers.

4. The apparatus of claim 3, wherein the bottom tray further comprises a bottom array of half chambers, such that when the bottom tray secures to a bottom of the photocatalytic filter bin, the upper array of the hemispherical half chambers in the bin floor mates with the bottom array of the half chambers and contains the photocatalytic material therein.

5. The apparatus of claim 1, further comprising means for securing the cover to the photocatalytic filter bin.

6. The apparatus of claim 1, further comprising at least one tanged tab downwardly protruding from the cover that secures to the photocatalytic filter bin.

7. The apparatus of claim 1, further comprising means for securing the bottom tray to the photocatalytic filter bin.

8. The apparatus of claim 1, further comprising at least one tanged tab upwardly protruding from the bottom tray that secures to a bottom of the photocatalytic filter bin.

9. The apparatus of claim 1, wherein the photocatalytic filter cartridge further comprises at least a portion of an electromechanical presence detector as an electrical safety mechanism.

10. The apparatus of claim 1, wherein the photocatalytic filter bin further comprises a downwardly protruding electrical switch engager as an electrical safety mechanism.

11. The apparatus of claim 1, wherein the photocatalytic filter cartridge further comprises optically transparent elements interspersed as light-distributors within the photocatalytic material.

12. The apparatus of claim 1, wherein the photocatalytic filter cartridge further comprises a uniquely-identifying QR code affixed to an exterior of the photocatalytic filter bin.

13. An apparatus for filtering and purifying air, comprising:
a photocatalytic filter cartridge inserted into an air purifier, the photocatalytic filter cartridge having an interior airflow path from an air inlet at a cover secured to a top of a photocatalytic filter bin, and the interior airflow path continuing through the photocatalytic filter bin to an air outlet in a bottom tray secured to a bottom of the photocatalytic filter bin, the photocatalytic filter cartridge including a pre-filter within in the photocatalytic filter bin downstream the interior airflow path from the air inlet cover, the photocatalytic filter cartridge including a secondary filter within the photocatalytic filter bin downstream the interior airflow path from the pre-filter, the photocatalytic filter cartridge including a photocatalytic material downstream the interior airflow path from the secondary filter and retained in the photocatalytic filter bin by the bottom tray;
a light source downstream the interior airflow path from the photocatalytic filter cartridge illuminating the photocatalytic material; and
a fan assembly downstream the interior airflow path from the light source;
wherein the fan assembly draws ambient air from the air inlet at the cover while the light source illuminates the photocatalytic material, and the fan assembly exhausts filtered and purified air exiting the photocatalytic filter cartridge.

14. The apparatus of claim 13, wherein the light source further comprises an upper array of light emitting diodes downstream illuminating the photocatalytic material included in the photocatalytic filter cartridge.

15. The apparatus of claim 14, wherein the light source further comprises a lower array of light emitting diodes upstream illuminating a separate, additional lower photocatalytic purification assembly arranged downstream from the photocatalytic filter cartridge.

16. The apparatus of claim 13, wherein the photocatalytic filter cartridge further comprises at least a portion of an electromechanical presence detector as an electrical safety mechanism.

17. The apparatus of claim 13, wherein the photocatalytic filter cartridge further comprises a downwardly protruding electrical switch engager, and the air purifier further comprises an electrical switch that senses a presence of the downwardly protruding electrical switch engager as an electrical safety mechanism.

18. The apparatus of claim 13, wherein the photocatalytic filter cartridge further comprises a uniquely-identifying QR code affixed to an exterior of the photocatalytic filter bin.

19. The apparatus of claim 13, wherein the photocatalytic filter cartridge further comprises transparent spheres mixed in with the photocatalytic material as light-distributing elements.

20. A method for servicing a photocatalytic filter cartridge, comprising:
retrieving, by a computer server, a QR code scanned from an exterior surface of the photocatalytic filter cartridge;
identifying, by the computer server, photocatalytic filter cartridge service records by querying a database having database entries that associate different QR codes to different photocatalytic filter cartridges including a database entry that maps the QR code to the photocatalytic filter cartridge service records;
determining, by the computer server, a photocatalytic filter cartridge cycle count associated with the photocatalytic filter cartridge based on the photocatalytic filter cartridge service records;
in response to the photocatalytic filter cartridge cycle count equaling or exceeding a threshold value, then rejecting, by the computer server, the servicing of the photocatalytic filter cartridge and sending a photocatalytic filter cartridge retirement notification; and
in response to the photocatalytic filter cartridge cycle count being less than the threshold value, then determining, by the computer server, that the photocatalytic filter cartridge is serviceable and queuing the photocatalytic filter cartridge for human servicing.
